(19) United States
(12) Patent Application Publication
(10) Pub. No.: US 2020/0209261 A1
(43) Pub. Date: Jul. 2, 2020

(54) DEVELOPMENT OF MICROBIAL BIOSENSORS FOR INTESTINAL INFLAMMATION

(71) Applicant: Baylor College of Medicine, Houston, TX (US)

(72) Inventors: Robert Allen Britton, Houston, TX (US); Jeffrey David Galley, US, TX (US)

(73) Assignee: Baylor College of Medicine, Houston, TX (US)

(21) Appl. No.: 16/612,590

(22) PCT Filed: May 11, 2018
(86) PCT No.: PCT/US2018/032457

§ 371 Date: Nov. 11, 2019

Related U.S. Application Data
(60) Provisional application No. 62/505,305, filed on May 12, 2017.

(51) Int. Cl. G01N 33/68; C12Q 1/02; C12Q 1/6897

(52) U.S. Cl. CPC G01N 33/6893; G01N 2800/065; C12Q 1/6897; C12Q 1/02

(57) ABSTRACT

Embodiments of the disclosure include systems, methods, and compositions for detection of imminent onset of a symptom of a gut inflammation medical condition. The disclosure also concerns microbial biosensors that detect a marker in the gut that is predictive of onset of at least one symptom of inflammatory bowel disease (IBD), for example, and such a sensor may include a promoter sensitive to the marker that is linked to expression of a detectable readout, such as in the feces of the individual with IBD.

### CROSS-REFERENCE TO RELATED APPLICATIONS

This application claims priority to U.S. Provisional Patent Application Ser. No. 62/505,305, filed May 12, 2017, which is incorporated by reference herein in its entirety.

### TECHNICAL FIELD

Embodiments of the disclosure include at least the fields of cell biology, molecular biology, bacteriology, gastroenterology, inflammation, diagnosis, and medicine.

### BACKGROUND

The inflammatory bowel diseases (IBD), which include Crohn's Disease (CD) and ulcerative colitis (UC), are chronic gastrointestinal (GI) disorders often associated with periodic symptomatic relapses. These episodes are caused by a hyperactive inflammatory response and the subsequent release of a cascade of damaging mediators [1]. Such flares are unpredictable in nature, and have a high probability of occurring on a yearly basis for IBD patients [2]. Compounding the difficulties associated with the erratic and disruptive nature of IBD symptomology are the current disease detection and maintenance options. Many of these methods, including endoscopy or magnetic resonance imaging (MRI), are invasive and costly. As a result of these negative aspects, they are not a realistic option for frequent diagnostic evaluations of IBD relapse. In recent years, researchers have identified calprotectin as a validated fecal marker for IBD, giving patients a cheaper option for disease monitoring [3]. Calprotectin is a neutrophil-source antimicrobial peptide that impinges upon bacterial growth through free metal chelation by sequestering zinc, manganese, and iron [4-6]. Fecal calprotectin assays identify concentrations above 100 ug/mL as being positive for GI inflammation, but also contain a range of borderline levels between 50 and 100 ug/mL calprotectin that require retesting due to lower predictive value [7, 8]. Herein lies a major issue, as compliance on retests can be low [9]. Thus, despite the reduced invasion and cost to the patient, fecal calprotectin detection can still miss the onset of a symptomatic flare due to missed retests on borderline results, as well as infrequent administration (3× per year) and primarily being used reactively to symptom onset instead of pre-emptively.

A need exists for an IBD monitoring method that is rapid and allows for more frequent observation and oversight by clinician and patient, and the present disclosure provides a solution for that long-felt need.

### BRIEF SUMMARY

Embodiments of the disclosure provide systems, methods, and compositions related to monitoring of a medical condition, including a gastrointestinal and/or inflammatory medical condition. In particular embodiments, the monitoring concerns a biological forewarning system for onset of one or more symptoms for patients with a gastrointestinal inflammatory condition, such as an inflammatory bowel disease (IBD). The system allows the patient to be notified before the onset of one or more particular symptoms of an IBD. In a specific embodiment, the system allows the patient to become aware of imminent onset of one or more symptoms without oversight by a medical practitioner. In at least certain cases, the system is sufficiently sensitive to detect biological signals of the medical condition in vivo before a symptom of the medical condition detectably manifests.

The systems, methods, and compositions of the present disclosure may be utilized in connection with any medical conditions that involve chronic inflammation of any kind. In some cases, the inflammation is of the digestive tract, a hallmark of IBD. Examples of particular IBDs include Crohn's disease and ulcerative colitis. In particular embodiments, the system detects a biological marker associated with a gastrointestinal symptom and the detection manifests in the feces of the individual. In specific embodiments, the detection of the biological marker occurs before manifestation of one or more symptoms from the inflammation occur. In at least some cases, the detection involves detection of a microbial biosensor that is sensitive to a fecal marker associated with IBD.

Embodiments of the disclosure include methods of determining a need for therapy for intestinal inflammation or cancer in an individual, comprising the steps of providing to the individual a population of non-pathogenic bacteria comprising an engineered polynucleotide, said polynucleotide comprising one or more direct calprotectin-sensor sequences or indirect calprotectin-sensor sequences operably linked to expression of a detectable readout product; and examining the feces of said individual for the detectable readout product. The calprotectin-sensor sequence may be a bacterial promoter. The sensor sequence may comprise part or all of one or more promoters from Lactobacillus reuteri 6475 and Escherichia coli Nissle 1917. In some cases, the calprotectin-sensor sequence comprises part or all of the L36/L31 ribosomal accessory protein promoter, part or all of the promoter for the enterobactin synthase, or both. The calprotectin-sensor sequence may comprise one or more zinc-uptake-regulator sites Any indirect calprotectin-sensor sequences may be directly or indirectly sensitive to a metal to which calprotectin binds, such as free zinc, iron, manganese, or a combination thereof.

In particular embodiments, the readout product is a detectable colorimetric or fluorescent marker. The readout product may be one or more of the following: violacein, one or more carotenoids, one or more phycobilins, one or more anthocyanins, and/or indigo. In specific cases, the readout product is green fluorescence protein, yellow fluorescent protein, blue fluorescent protein, mCherry, or cyan fluorescent protein.

In certain embodiments, the providing step occurs orally and may be performed by the individual. The providing step may or may not occur on a regular basis. It may occur during the presence or absence of one or more symptoms of intestinal inflammation. In specific cases, when the level of calprotectin in the gastrointestinal tract of the individual is ≥100 ug/mL, the readout product is detectable. The examining step of the feces for the detectable readout product may or may not be performed by the individual. In some caseswhen the readout product is detected, the individual obtains treatment for the intestinal inflammation, which may or may not be from inflammatory bowel disease (IBD), including Crohn's Disease (CD) or ulcerative colitis (UC), as examples. In some cases when the readout product is detected, the individual receives treatment of the inflammation or cancer prior to onset of one or more symptoms.

In one embodiment there is a non-pathogenic bacteria or population thereof comprising an engineered polynucleotide, said polynucleotide comprising one or more direct calprotectin-sensor sequences or indirect calprotectin-sensor sequences operably linked to a sequence that encodes a detectable readout product. The calprotectin-sensor sequence is a bacterial promoter, in some cases and may comprise part or all of one or more promoters from Lactobacillus reuteri 6475 and Escherichia coli Nissle 1917. The calprotectin-sensor sequence may comprise part or all of the L36/L31 ribosomal accessory protein promoter, part or all of the promoter for the enterobactin synthase, or both. The indirect calprotectin-sensor sequences may be directly or indirectly sensitive to a metal to which calprotectin binds, such as free zinc, iron, manganese, or a combination thereof. In particular embodiments, the readout product is a detectable colorimetric or fluorescent marker. The readout product may be one or more of the following: violacein, one or more carotenoids, one or more phycobilins, one or more anthocyanins, and/or indigo. In some cases, the readout product is green fluorescence protein, yellow fluorescent protein, blue fluorescent protein, one or more phycobilins, one or more anthocyanins, or cyan fluorescent protein.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawing, in which:

FIGS. 1A and 1B illustrate an embodiment of the disclosure. 1A) Without an inflammatory signal, the synthetic induction sensory pathway is inactive. 1B) Upon sensing of inflammatory signal, the promoter is activated, turning on violacein production;

FIGS. 2A and 2B show fold-increase in GFP expression when co-cultured in 40 ug/mL calprotectin. 2A) L36/L31 Ribosomal Accessory Promoter; and 2B) Enterobactin Synthase Promoter; Data collected over 3 or 4 individual runs, all data Mean +/−SD;

FIGS. 3A and 3B show fold increase in GFP expression when co-cultured with single IBD sample. 3A) L36 Ribosomal Accessory Promoter; 3B) Enterobactin Synthase Promoter; Duplicate technical replicates; All data mean +/−SD;

FIG. 4 demonstrates minimum inhibitory concentration of calprotectin on candidate microbes;

FIG. 5 shows calprotectin induction on candidate microbes;

FIG. 6 demonstrates N,N,N′,N′-tetrakis(2-pyridinylmethyl)-1,2-ethanediamine (TPEN) induction on candidate microbes;

FIG. 7A shows Manganese addition with E. coli Nissle and FIG. 7B shows Manganese addition with Lactobacillus reuteri;

FIG. 8A provides data for Zinc addition with E. coli Nissle, whereas FIG. 8B provides data for Zinc addition with L. reuteri; and

FIG. 9A shows Iron addition with E. coli Nissle and FIG. 9B shows Iron addition with L. reuteri.

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially” of or “consist of” one or more sequences of the invention, for example. Some embodiments of the invention may consist of or consist essentially of one or more elements, method steps, and/or methods of the invention. It is contemplated that any method or composition described herein can be implemented with respect to any other method or composition described herein.

### I. General Embodiments

Microbes can be agents of biomarker sensing [11, 12]. Use of microbial biomarker detection has been accomplished in murine liver tumor metastasis detection and human urine glucose levels [11, 12]. However, while these studies provide proof-of-concept that microbial biosensors of health-associated biomarkers are practical, the early advances in diagnostics require trained lab staff for sample analysis. An at-home monitoring would produce an output that could be measured without equipment, for example through the alteration of stool color. Shifting IBD detection from the clinician's office to the home would greatly reduce overall clinical visits, giving patients an easier, speedier method of symptom monitoring. This system will allow greater patient autonomy and symptom predictability as well as reduced medical costs, residual effects that could improve quality-of-life. As living with IBD becomes commonplace, an emphasis must be placed on developing technologies that can alleviate day-to-day battles with the disease. Microbial biosensors for IBD would turn IBD monitoring and treatment into a self-managed system similar to the current paradigm for diabetes monitoring, with in-home insulin treatment. This system illustrates the efficacy of synthetic microbial detection systems. As disease biomarkers are identified and corroborated, this system may be utilized as a ‘plug-and-play’ backbone for a variety of detection compositions (for example, expression constructs), particularly in microbe-accessible regions.

Embodiments of the disclosure include microbial biosensors for inflammation detection in an individual, including at least intestinal inflammation. Detection of the microbial biosensor provides clinical information for the individual, including onset of inflammation itself or any symptom(s) related to inflammation in general or an IBD specifically. The microbial biosensor in at least some cases provides clinical information that is rapid, non-invasive, and is utilized in real-time, including in an at-home setting, as an example. Such a use reduces the need for clinical contact and facilitates patient compliance. In at least some cases, routine use of the system, including repeated administrations, facilitates reduction of day-to-day variance.

In specific embodiments, the biosensors are sensitive to one or more inflammation biomarkers, including one or more gut inflammation biomarkers. The disclosure provides for a microbial (including bacterial) biosensor that senses an inflammatory level of at least one disease biomarker and produces a detectable output based on the presence of the disease biomarker(s). In at least some cases, the inflammatory level of one or more disease biomarkers is recognized based on inflammation-induced promoters in a diagnostic gene expression system. In specific embodiments, a detectable output based on inflammation biomarker-induced gene expression of a detectable gene product is interpreted by the individual with the disease. In specific cases, the detectable output comprises a detectable characteristic of the individual's feces, such as a change in color or the presence of fluorescence, for example. As an example, a change in color may be a detectable dye pigment in the feces, or fluorescence in the feces may be detected based on suitable light conditions.

In certain embodiments, the microbial biosensor system detects a particular compound associated with the gut inflammation disease. As an example, the microbial biosensor system may detect a compound secreted by neutrophils related to IBD. As a further example, the microbial biosensor system may recognize a secreted antimicrobial peptide from neutrophils, such as calprotectin that is a heterodimer with each peptide having specificity to certain metals. Calprotectin makes up approximately 50% of total neutrophil granule proteins, is bacteriostatic, and sequesters zinc, manganese, and iron. Thus, in specific embodiments calprotectin sensitivity is associated with an IBD biomarker in fecal testing. In some cases, calprotectin is employed in the context of the methods of the disclosure as being a marker for any kind of inflammation. In alternatives, a bacterial-sensitive inflammatory biomarker other than calprotectin is employed.

In particular embodiments, medical conditions associated with high calprotectin levels are detected utilizing methods of the disclosure. In specific cases, Clostridium difficile infection results in high calprotectin levels and may be the subject of methods for detecting onset of one or more symptoms.

In particular embodiments, the disclosure concerns the development of an in-home inflammation monitoring system that would introduce a vast improvement for IBD flare detection, giving patients advanced warning that currently is not represented in IBD diagnostics. Embodiments include a synthetic probiotic that detects and reports particular calprotectin levels (for example, at >100 ug/mL). In one embodiment, the detection occurs through the linking of microbial promoters (including aerobic or anaerobic bacterial promoters) to the secretion of a detectable pigment. In specific embodiments, calprotectin-sensitive promoters are utilized. In specific embodiments of calprotectin-sensitive promoters, promoters of genes are utilized that are upregulated greater than at least two-fold by calprotectin induction (but below levels of calprotectin that are inhibitory to bacterial growth).

The microbial biosensor may be taken orally by a patient at home on a regular schedule, allowing the patient to monitor disease state in real-time on a much shorter timescale, with increased frequency of diagnostic administrations. This inflammation biosensor may be engineered to have total repression of signal in the absence of stimulating calprotectin. In the presence of inflammatory levels of calprotectin anywhere in the gastrointestinal tract, production of the detectable readout is initiated, and then sustained and amplified over the course of passage throughout the GI tract. Positive signal is detected in excreted stool within days of taking the probiotic biosensor, giving the patient an earlier warning of potential inflammatory onset.

Examination of the feces for the detectable readout product may occur by any suitable method and in at least some embodiments is performed by the individual having the gastrointestinal inflammatory condition. In some cases a medical practitioner may make the determination of the presence of the detectable readout product or may confirm the determination by the individual. In specific embodiments, the examination is visual and requires no manipulation of the feces. In other cases, the examination is visual and includes manipulation of the feces. For example, one may be required to manipulate the feces in order to detect the detectable readout, such as when a region of the feces in which the detectable readout product is present is internal within the feces and obscured to the naked eye. In specific embodiments, the toilet or receptacle in which the examination step is made comprises one or more compounds in the water that allows, facilitates, or enhances visualization of the detectable readout.

In embodiments of the disclosure, there is a method of determining a need for therapy for intestinal inflammation or cancer in an individual, comprising the steps of providing to the individual a population of non-pathogenic bacteria comprising an engineered polynucleotide comprising one or more direct calprotectin-sensor sequences or indirect calprotectin-sensor sequences operably linked to expression of a detectable readout product; and examining the feces of the individual for the detectable readout product.

In some embodiments, there is a method of monitoring a gastrointestinal inflammatory condition in an individual, including monitoring for the onset of one or more symptoms of the gastrointestinal inflammatory condition.

In some cases, there are methods of monitoring a therapy for a gastrointestinal inflammatory condition for an individual. The individual is provided a population of non-pathogenic bacteria comprising an engineered polynucleotide that comprises one or more direct calprotectin-sensor sequences (or indirect calprotectin-sensor sequences operably linked to expression of a detectable readout product. Following onset of one or more symptoms of the gastrointestinal inflammatory condition, the individual detects the detectable readout product upon examination of their feces. The individual is provided one or more therapies for the gastrointestinal inflammatory condition and continues over time to examine their feces for the detectable readout product. When the therapy is providing treatment of one or more symptoms of the gastrointestinal inflammatory condition, the detectable readout diminishes, including in some cases to a non-detectable level.

In particular embodiments, the microbial biosensor system detects calprotectin at particular levels in the gut. The level in specific cases may be >100 ug/mL, >110 ug/mL, >120 ug/mL, >125 ug/mL, >130 ug/mL, >140 ug/mL, >150 ug/mL, >175 ug/mL, and so forth, for example. In some cases, the system is suitable to detect calprotectin at levels <100 ug/mL, such as when a certain calprotectin-sensitive promoter is used or when the system is engineered to have enhanced detection. However, in certain cases the system is useful only when the level of calprotectin is at a certain level (for example, >100 ug/mL), for example, so that false positive readings are avoided.

In particular embodiments, the microbial biosensor is ingested as non-pathogenic bacteria that comprise an engineered polynucleotide that has one or more direct calprotectin-sensor sequences or indirect calprotectin-sensor sequences operably linked to expression of a detectable readout product. Such non-pathogenic bacteria may be ingested by the individual on a routine basis so that the individual is ensured of detecting the presence of the detectable readout in their feces as it occurs and also to expose the individual to the practice and habit of monitoring their feces and becoming familiar with its day-to-day appearance. In specific embodiments, the bacteria are ingested every day, once or twice a day, every other day, once a week, one to three times a week, several times a month, and so forth. The bacteria may be ingested 1-2, 1-3, 1-4, 1-5, or 1-6 times a week, in some cases.

The detectable readout of the system may be detectable in a variety of ways, as an example so long as an individual is not required to employ a medical practitioner for making the determination. In some cases, the detectable readout in the feces comprises a color change in the feces. The color may be of any color so long as it is distinguishable from the feces color. In other cases, the detectable readout in the feces comprises the presence of fluorescence, and such a determination may require particular light conditions to be able to identify the fluorescence (for example, turning off overhead or other lights in the room or using a fluorescence detection device).

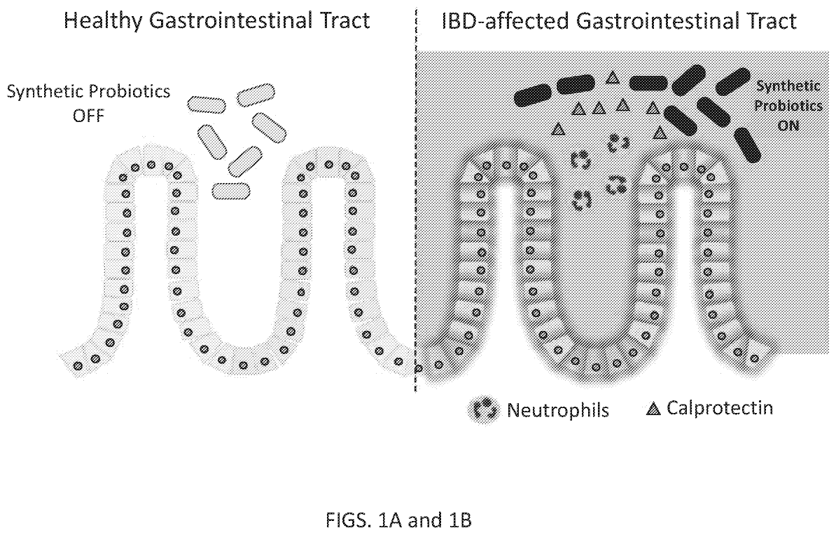

In specific embodiments, the detectable readout is a specific pigment in the feces, such as a non-toxic pigment. In specific cases, one can utilize violacein, a non-toxic bacterially-derived pigment, although alternatives to violacein include anthocyanins, indigo and/or one or more carotenoids (for example, α-carotene, (β-carotene, and/or lycopene) and one or more phycobilins (for example, phycocyanobilin). Violacein production will shift fecal color, turning stool a purple hue that would be visible to the patient upon excretion (FIG. 1).

As an illustrative embodiment only, this disclosure encompasses a microbial biosensor that utilizes endogenous promoters of Lactobacillus reuteri PTA 6475 and Escherichia coli Nissle 1917 to detect the presence of calprotectin, a neutrophil-source antimicrobial peptide. The primary function of calprotectin is to sequester zinc, iron, and manganese from the extracellular space. The endogenous promoters that are being used in the sensors have all demonstrated sensitivity to zinc deficiency and the metal-binding properties of calprotectin. The biosensors may have their sensing of calprotectin-induced zinc deficiency coupled with the expression of a colorimetric dye, violacein. The dye production may be enhanced to alter fecal pigment, giving patients a private and in-home method of monitoring and detecting intestinal inflammation. Further, the biosensors may be optimized to detect calprotectin at a level of at least 100 μg/mL, which is indicative of inflammation in human gastrointestinal tracts.

In particular embodiments, upon detection of the detectable readout in the individual's feces, the individual may take action to treat one or more symptoms of the gut inflammation. The action(s) may reduce the intensity of the symptom or delay the onset of the symptom. Examples of treatment include one or more anti-inflammatories, one or more antibiotics, one or more Aminosalicylates (5-ASAs), one or more corticosteroids, one or more immune modifiers (immunomodulators), and/or one or more biologic therapies. Specific compounds include metronidazole, ciprofloxacin, sulfasalazine, mesalamine, olsalazine, balsalazide, prednisone, azathioprine, cyclosporine, 6-mercaptopurine, tacrolimus, methotrexate, infliximab, infliximab-dyyb, or a combination thereof.

### II. Identification of Calprotectin-Sensitive Promoters

Further calprotectin-sensitive promoters than those described specifically herein could be identified through RNA sequencing technology. Different concentrations of calprotectin could be used, as well as different incubation periods. Also, RNA sequencing could be performed on potential probiotic bacteria in addition to E. coli Nissle and L. reuteri (including, but not limited to, Lactococcus lactis). Potential microbes could also be co-cultured in fecal slurries obtained from patients with inflammatory bowel disease, and RNA sequencing can be performed on RNA isolated from these microbes in order to find IBD-specific promoters.

As an example of a culturing/flow protocol, sensors are incubated for 4-6 hours with either an induction agent or fecal slurry using minimal media. Growth to early log phase (about 5 doublings) then occurs. Cells are then diluted into flow sheath fluid and GFP is measured via flow cytometry.

### III. Calprotectin-Sensitive Promoters

The disclosure includes embodiments wherein calprotectin-sensitive promoters are utilized as a means for detection of calprotectin at a level that signals the onset of one or more symptoms of gut inflammation. In particular cases, binding of calprotectin to a bacterial cell elicits activation of a bacterial promoter resulting in the expression of a gene product that is detectable, such as detectable in feces. Alternatively, calprotectin alters the environment in such a way that elicits activation of a bacterial promoter resulting in the expression of a gene product that is detectable, such as detectable in feces. One such way that calprotectin alters the environment is by chelating metals, which may alter expression of calprotectin sensitive promoters via depletion of metals such as Zinc, Iron, or Manganese.

Thus, upon identification of a calprotectin-sensitive promoter, the promoter may be operably linked to a polynucleotide encoding a readout gene product that is detectable. The detectable gene product may produce a product that is colorimetric, fluorescent, or light-sensitive. An expression construct comprising a calprotectin-sensitive promoter operably linked to expression of a polynucleotide encoding a detectable readout gene product may be utilized. For example, the expression construct may be located within a vector, such as an expression vector, lentiviral vector, adenoviral vector, or adeno-associated viral vector.

In some cases, a calprotectin-sensitive promoter is derived from a bacterial genome. Such a promoter may be utilized in its entirety, or the promoter may be modified compared to the endogenous bacterial promoter sequence. For example, a bacterial promoter may be truncated to modify the strength of the promoter or to reduce background expression levels of the promoter. Such modifications may increase the signal to noise ratio of the promoters, allowing for increased sensitivity.

In specific embodiments, the promoters are from E. coli Nissle or Lactobacillus reuteri. As particular embodiments, promoters in L. reuteri 6475 and E. coli Nissle 1917 that are sensitive to IBD-associated biomarker(s) are utilized.

Examples of specific calprotectin-sensitive promoters are as follows:

E. coli Nissle L36 Accessory Protein Promoter-
ykgMO
(SEQ ID NO: 1)
taacggcaataaactgttcacttcagTGATATTTAAAATATGCATCCTCT
CCCTTTTTTGTAAGTAATTATTATATCCGTGGGAGAGGAATACACATTGT
CAGGTAATCAATCATGCTGCAATAAATCATCGGCCAGTAAAGTGGAGATA
GCCTCCATTCTCGAAAAATCCATACTCTCAGCGAAACCATCATCAATCAC
TCATCCAGGCGTTTATGGGAGCGTCGCCAATGGCTGCTAACAATGCCAGA
CTTCCCCGTTGCGGAAATTCCACATCCCACAAATAGTCACAGTGATTGGG
TGTTGAAATGATCCGGATGAGCATGTATCTTTAcggttatgttataacat
aacaggtaaaaatg
https://ecocyc.org/gene?orgid=ECOLI&id=G6167#
tab=TU
E. coli Nissle Enterobactin Synthase Promoter-
entCEBA-ybdB
(SEQ ID NO: 2)
aagtcagatcctgttattaatgaagTTAATGCTTCTCATTTTCATTGTAA
CCACAACCAGATGCAACCCCGAGTTGCAGATTGCGTTACCTCAAGAGTTG
ACATAGTGCGCGTTTGCTTTTAGGTTAGCGACCGAAAATATAAATGATAA
TCATTATTAAAGCCTTTATcattttgtggaggatgat
https://ecocyc.org/gene?orgid=ECOLI&id=EG10261#
tab=TU
E. coli Nissle ABC Transporter Promoter-
(SEQ ID NO: 3)
aacagacccgaagaaaatgaaatataagAAAAGATCAACGAGTGAAGAAA
AAGTTCAAAAAATGGCTGCCGGGGAGGAAGGAAAGTACCGGATGGAAAGA
GTCCCCCCTAAAGCAGACTGACAGACATAACAAATCCCCGGGGGGATTTG
TGTATAAGAGACAGTACTTATCTGGAGGTAATTGCAATATCTCTGTGAAC
TTACACAGGGTGGGCTTACCGCATACACTGACACTTAGCGGATCGACAGA
ACATTATTAACAGAGCATCACTGAACGCTACATAATCAGAGTTGCATAAA
TAAAATGTTATTAACATcacaatcacaacatttcga
L. reuteri Acyl Carrier Protein Dehydratase-
(SEQ ID NO: 4)
Ttaataacgagcaaatattattttaccaattctcaattaaatagtaaagg
aatattttaatttaaggtgattaagaattaaatgacataaaaataatttg
acattcaaaatatttttgaatataatttgatcatcgaatattttgatact
cgaaatatttttttgaaggcaggtgaatcttt
L. reuteri ABC Transporter Promoter-
(SEQ ID NO: 5)
Tcatatttaaggacaagaggatggaaagaagtaactttctctcttgtcct
ttttatttttatgttgcaatcccgcataaagttcgatattatattcattg
ttctaaatagtaacgattacgattaaaaagagggaaaga

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Microbial Biosensors for Gut Inflammation

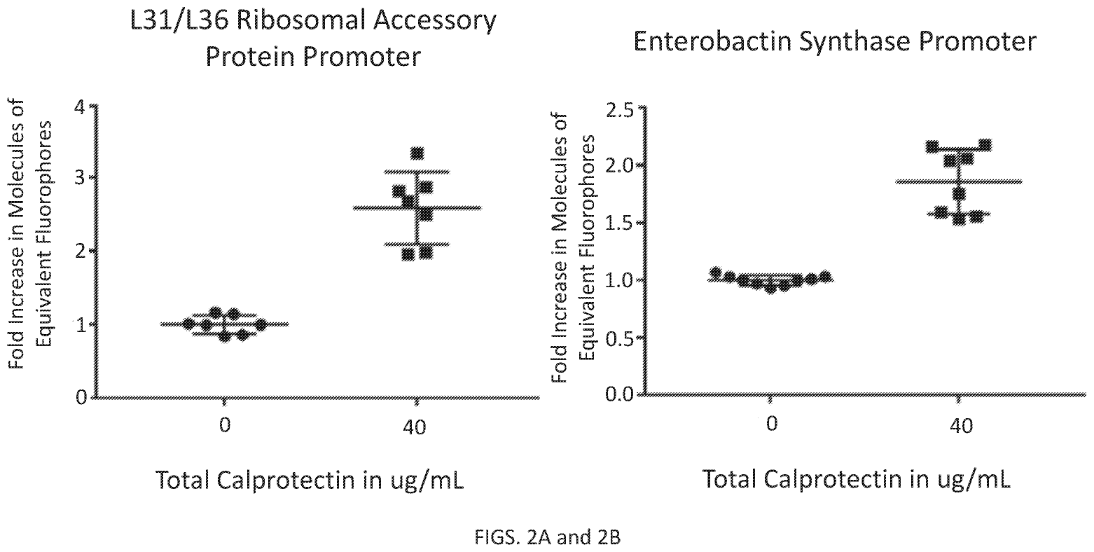

Initial Data. RNA-seq was performed on E. coli Nissle 1917 grown in the presence of sub-inhibitory levels of recombinant human calprotectin to identify genes that are up- and down-regulated by the presence of the peptide. Sequences were obtained via Illumina NextSeq and mapped against E. coli Nissle 1917 reference genome. Promoter regions of genes up-regulated at least two-fold were copied from the E. coli Nissle 1917 chromosome and ligated upstream of a GFP cassette on a pColE1 plasmid before being cloned back into E. coli Nissle 1917. Functionality of the raw promoter constructs were verified by co-culturing with purified calprotectin and then measuring GFP output via flow cytometry. Though multiple promoter constructs exhibited sensitivity to calprotectin, sensor development was focused on two specific promoters: the L36/L31 ribosomal accessory protein promoter, involved in ribosome stabilization and the promoter for the enterobactin synthase, a siderophore. The former exhibits a ˜2.6 fold increase in GPF expression in the presence of 40 ug/mL calprotectin compared to un-induced levels, while the latter exhibited a ˜1.8-fold increase in GFP. Thus, even prior to any promoter engineering to elevate dynamic range, the promoter constructs are able to respond to calprotectin induction measurably higher than the un-induced state (FIG. 2A-2B). In addition, these promoter constructs represent two different baseline reporter output levels, as L36/L31 expresses at a high level in the absence of calprotectin induction and enterobactin synthase at a low level. Both promoters are sensitive to zinc chelation, as evidenced by the use of synthetic zinc chelator, TPEN. GFP expression in the L36/L31 and enterobactin synthase promoter constructs was increased 4.5-fold and 5.6-fold respectively when co-cultured with 1.5 uM (N,N,N′,N′-tetrakis(2-pyridinylmethyl)-1,2-ethanediamine (TPEN) (equivalent to 40 ug/mL calprotectin) compared with media only. Furthermore, adding zinc to sensors co-cultured with calprotectin abolishes the calprotectin-induced increase in GFP expression. As the RNA-seq data and these results show, E. coli Nissle responds to calprotectin and TPEN by turning on zinc-starvation related promoters, unsurprising given calprotectin's primary function is to chelate free zinc. E. coli Nissle has co-evolved with human hosts, so it is likely that this response has developed as a natural response to gastrointestinal calprotectin release, strengthening the case for the use of E. coli Nissle as a biosensor for GI inflammatory disease. Zinc deficient regions in inflamed guts are likely common due to the multitudinous release of calprotectin and utilizing the natural response of E. coli Nissle for biosensor function will be key in proving practicability.

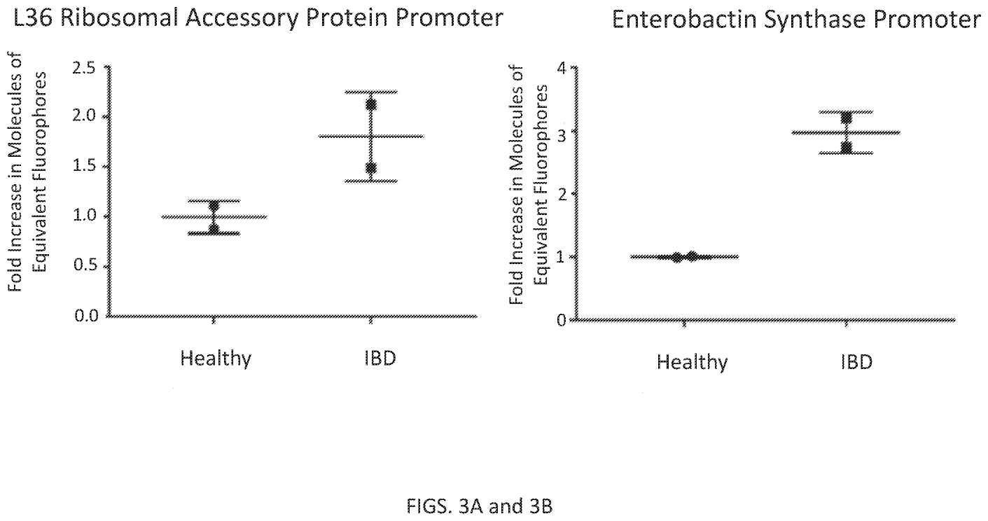

When co-cultured with a fecal sample obtained from an IBD patient that contained 1800 ug/mL calprotectin, the raw sensors produced a 1.8-fold (L36 ribosomal promoter) and 3.0-fold (enterobactin promoter) increase in GFP expression compared to those co-cultured with a sample that had 49 ug/mL calprotectin (FIG. 3A-3B). Notably, though the sensors were activated in 40 ug/mL in the purified calprotectin assays, a healthy fecal slurry in that range did not induce GFP expression. This is likely due to the background zinc levels being lower in the M9 medium used for the assays compared to gut zinc levels, leading to a differential point at which the sensors become active.

In one embodiment, a synthetic biosensor is engineered for the IBD-associated biomarker calprotectin. In specific cases, optimization of synthetic biosensors is achieved using the probiotic, E. coli Nissle 1917, a generally regarded as safe microbe that can survive well in the GI tract and is highly malleable to precision genome editing techniques [13, 14]. In specific cases, these sensors are in a repressed OFF state when local calprotectin levels are <100 ug/mL, and upon sensing of >100 ug/mL fecal calprotectin, promoter activity is augmented for maximal reporter output. Optimal augmentation may be represented by a sensor that stays on throughout the GI tract regardless if ≥100 ug/mL calprotectin is only sensed as high as the jejunum, as well as by a sensor that secretes an observable level of violacein that alters fecal pigment. Calprotectin-sensitive E. coli Nissle biosensors may be synthesized using calprotectin-induced promoters identified through RNA-seq, and verified their function in raw, pre-optimized form against both purified calprotectin and fecal slurry (FIGS. 2, 3). One can utilize a variety of available synthetic and molecular biology tools to increase reporter output in the presence of ≥100 ug/mL fecal calprotectin induction and reduce baseline reporter output expression in the calprotectin-absent state. One can confirm the functionality of the engineered biosensors in ex-vivo and in-vivo models of both complex microbial communities and intestinal inflammation.

Calprotectin-sensitive promoters may be engineered for optimal signal strength. Having now established two working E. coli Nissle sensors that are sensitive to calprotectin, one can optimize reporter output. An optimized sensor may be characterized as a sensor that expresses GFP in fecal slurries that contain ≥100 ug/mL calprotectin, with abolished output below that level. Though the sensors already express elevated levels of GFP in both purified recombinant calprotectin as well as a fecal sample from an IBD patient, increasing the overall dynamic range of the promoters can increase the signal to noise ratio, and can better correlate with greater violacein output. Thus, one can increase promoter strength and ultimately GFP output in response to calprotectin induction while concomitantly reducing background of prospective promoters to an OFF state in the absence of calprotectin.

Approach. A number of available methods to increase the dynamic range and fine-tune the sensitivity of the promoters may be pursued. First, one can perform promoter truncations, which can result in reduced background and greater induction [10]. Though both promoters are well-characterized, the existence of undiscovered genetic features may impede expression during induction. Thus, promoters may be truncated in 25 bp sections from both the 3′ and 5′ ends, and GFP expression levels may be evaluated on the truncated-promoter constructs after co-culture with calprotectin. One can also reduce the high background leakiness of the L36/L31 promoter. The promoters are sensitive to the zinc chelation function of calprotectin, as evidenced by the activation of the promoters in the presence of TPEN and the abolishment of calprotectin-induced signal when zinc is added. By adding zinc-uptake-regulator (zur) sites in the promoter region, one can affect promoter leakiness. Zur sites are docking regions for regulators that act as repressors on gene transcription when bound to a zinc ion during periods of ample zinc availability. During metal starvation, the regulators no longer repress upon losing its bound ion, and downstream genes can be transcribed [15]. The L36/L31 intergenic promoter region has a single zur site. Research has shown that the L31 gene is particularly sensitive to zinc deficiency, and would be one of the earliest genes transcribed in the event of zinc chelation [16]. Thus, it is possible even slight perturbations in zinc levels could lead to leaky transcription. By cloning extra zur sites into the intergenic promoter sequence, one can reduce promoter leakiness by increasing the total repressor-operator sites for Zur repression on downstream gene transcription. Synthetic biology research has demonstrated that increasing binding sites for repressors can reduce baseline transcription, while leaving maximum outputs mostly unchanged in induced states [17].

In particular embodiments, a major advantage of the biosensor is to detect inflammation in the early stages, in order to give early warning of a symptomatic flare. Thus, one can modulate promoter activity for sensitivity to 100 ug/mL fecal calprotectin. To affect promoter sensitivity to calprotectin concentration, the aforementioned methods of truncation and zur site manipulation may be used. One can also perform site-directed mutagenesis and −35/−10 consensus sequence engineering in order to increase or decrease promoter binding to create a range of promoter strengths [18]. Lastly, one can associate promoter activation in high-calprotectin IBD samples with zinc deficiency by supplementing IBD fecal slurries that contain high levels of calprotectin with zinc and co-culturing these supplemented slurries with the sensors. In specific embodiments, this will ablate the calprotectin-induced GFP expression, demonstrating that the specific activation of the promoters in these samples is due to zinc deficiency. All optimization tests may be performed on fecal samples collected from both IBD and healthy patients.

There was successful identification and validation of two calprotectin-sensitive E. coli Nissle promoters when cultured with either purified calprotectin or an IBD fecal slurry. One can develop an engineered calprotectin-sensitive biosensor that will have robust GFP output in the presence of inflammatory levels of calprotectin and reducing baseline in the L36/L31 promoter closer to a ‘white-cell’ level, without affecting the high GFP expression when induced with calprotectin seen with the raw promoter.

In specific embodiments, one can include a repressor-operator systems such as lacI. Herein, a variable strength constitutive promoter can be placed upstream of a repressor (e.g. lacI, tetR) and the specific operator placed directly upstream of the calprotectin-sensitive promoter. One can tune promoter sensitivity through the strength of the constitutive promoter [19]. Weak promoters would weakly repress transcription of the calprotectin-sensitive promoter through lower repression while the use of a strong constitutive promoter would increase sensitivity to a greater extent through greater repression. Manipulating the spacing between either the consensus sequences and the +1 site, or the RBS site also affects transcriptional strength and may be utilized.

One can validate and optimize calprotectin-sensitive biosensor function using in vivo modeling. Engineered L36/L31 and enterobactin synthase promoters that have exhibited maximum dynamic range through the promoter engineering methods may be utilized. Here, the engineered sensors may be tested using both mini bioreactor assays (MBRAs) to model sensor activity in complex microbial communities and in in vivo inflammatory models in order to evaluate overall signal strength, as well as signal longevity and full characterization of signal output through the murine gastrointestinal tract.

In order to investigate how the sensors would behave in a complex gut environment, one can first examine sensor response in MBRAs, a high throughput model for microbial interactions in a gut-like environment of complex microbial communities [20]. Calprotectin may be added to the MBRA in addition to the biosensor. Samples may be collected periodically and GFP production may be quantified via flow cytometry. Next, one can utilize in-vivo models to evaluate the sensor in an inflamed GI tract. One can use two separate outputs to evaluate sensor function: GFP as well as a luciferase reporter, in order to take advantage of the in-vivo imaging system (IVIS) and to inform on the lifespan of the signal. Luciferase reporters provide a far more sensitive readout, and allow one to locate where along the gastrointestinal tract the sensors are sensing calprotectin and activating [21]. In particular cases, the sensor behaves in environments of localized higher inflammation, as seen in Crohn's Disease, and multiple murine inflammatory models may be used: Dextran sodium sulfate (DSS), Citrobacter rodentium, and Toxoplasma gondii. For chemically-induced colitis, C57BL/6 mice may be exposed to 3% DSS over the course of 7 days [22]. At the peak of inflammation on day 7, optimized sensors may be gavaged into both DSS-treated mice and water controls. One can also use the Citrobacter rodentium A/E colitis model to ensure promoter activity is not specific to DSS-induced colitis [23]. The general outline may be similar, though infection severity peaks between days 9 and 12, meaning mice may receive the optimized biosensors on the mornings of days 9 through 12. Lastly, one can utilize the Toxoplasma gondii model, which is one of the few IBD models that cause severe small intestinal inflammation [24, 25]. In embodiments, one ensures that signal activation is not short-lived and can be measured after passage from small intestine through colon and to excretion. To build the luciferase detection system, the luxCDABE luciferase gene cassette may be cloned downstream of the calprotectin-sensitive promoters in place of GFP, and signal transduction in pure culture/fecal pellets may be initially verified by measuring luminescence on a luminometer. One can evaluate specificity of signal while in transit in a mouse, as well as the time-scale over which it is active. In all studies, colonic, cecal, and small intestinal contents as well as feces may be collected over the course of sensor passage through the GI tract and processed, as published previously [10]. GFP expression/luciferase activity from the sensors may be analyzed via flow cytometry or luminometer. Reporter output may be compared via appropriate pairwise (T-test) and multi-level (ANOVA) analyses. For all in vivo studies, non-infected controls may be used to ensure specificity of the biosensor for inflammatory microenvironments.

In certain embodiments, there is an observable and significant increase in GFP expression in MBRAs seeded with calprotectin compared with control MBRAs, as well as measurable increases in reporter output when examined in in vivo inflammatory models. Positive signal in inflamed mouse guts with a concomitant negative signal in healthy mouse guts may indicate that the sensors have a specific response to GI inflammation. In addition, measurable signal in the T. gondii model upon excretion is useful, as this is a major indication that the sensor is active over longer periods of time, as T. gondii infection is focused upon the small intestine.

In the case that the sensor response is short-lived and is not sustained throughout the entirety of the passage of the GI tract, one can engineer positive feedback loops, such as the LuxllLuxR quorum sensing unit to increase reporter output in response to calprotectin-sensing so that when the signal turns on, it stays on [26]. If there are any issues regarding the function of E. coli Nissle in the GI tract, one can also develop a calprotectin biosensor using gram positive Lactobacillus reuteri as a vector. As with E. coli Nissle, these sensors have shown efficacy as calprotectin sensors and may be used as an alternative.

One can develop a dye-based microbial detection method of calprotectin by engineered-promoter biosensors. One can combine the calprotectin-sensitive microbial biosensor with the production of a dye that can be utilized for in-home inflammatory detection by patients. One can focus on utilizing the violacein dye (as an example) to be produced by recombinant E. coli Nissle as the reporter mechanism. This system may allow for expression of the violacein molecule upon detection of calprotectin. One can guarantee the feasibility of violacein production as a fecal pigment stain and ensure its function in murine gastrointestinal tracts. One can associate the optimized calprotectin-sensitive promoter system with violacein output for validation in MBRA and in vivo inflammatory models.

One can validate the feasibility of recombinant violacein synthesis and secretion in both ex vivo and in vivo models. One can determine if violacein is a viable candidate for fecal dye staining from microbial sensing of calprotectin. Violacein is a violet shaded pigment that is produced in nature by multiple bacterial strains, including Chromobacterium violaceum [29]. Production is encoded by the vioABCDE operon, which starts with the L-tryptophan precursor, culminating in violacein. The operon has been successfully ported into recombinant microbes including Citrobacter freundii and Escherichia coli, and pigment production has been optimized through metabolic engineering in these species [28]. One can validate violacein as the reporter gene in a calprotectin biosensor.

vioABCDE may be cloned into E. coli Nissle under the control of constitutive and inducible promoters. One can use the LacI inducible system in E. coli Nissle to evaluate differential violacein levels under varying IPTG induction concentrations, focusing on the effect on growth rate and total violacein output. Dye production may be measured after ethanol extraction from recombinant bacteria cultures by spectrophotometry [28], and bacterial growth may be measured via plating. After determining optimal induction level in E. coli Nissle that does not impede bacterial growth rates, one can clone the violacein operon into a plasmid under the control of an optimal strength constitutive promoter using the defined-strength Anderson promoter library. After verification of production in vitro, one can gavage C57B1/6 mice with constitutively-expressed violacein-producing E. coli Nissle to determine the amount of violacein production required to alter the fecal pigment to an observable purple hue.

Maximally producing violacein in E. coli Nissle without affecting bacterial growth can be achieved. One can verify violacein production in vitro. One can measure observable violacein signal observed in fecal output from mice after gavage with the pigment-producing E. coli Nissle.

In embodiments wherein violacein as a colorimetric dye interferes with bacterial viability, one can utilize multiple microbe models and can expand further to Lactobacillus reuteri. Likewise, alternative options for dyes may be utilized, including carotenoids and indigo, which have also been manipulated for production in recombinant bacteria [29, 30].

One can assess and optimize violacein production by calprotectin-responsive microbial biosensors in presence of in vitro and in vivo inflammation. One can ensure that the sensor stays on through GI passage, producing violacein in response to both large and small intestinal inflammation, in certain embodiments.

The violacein operon, vioABCDE, may be cloned in place of the GFP gene cassette in the engineered biosensors described elsewhere herein. After the synthetic biosensor-violacein system has been synthesized, violacein production from the E. coli Nissle biosensors may initially be evaluated in the presence of calprotectin in vitro. In addition, sensors may be co-cultured with IBD and healthy fecal slurries containing inflammatory and non-inflammatory levels of calprotectin in order to evaluate violacein output. Next, the biosensors may be cultured in MBRAs to verify the efficacy of the recombinant probiotic to produce and secrete violacein in response to calprotectin while also in the presence of a complex microbial community. The biosensors may be tested using in vivo inflammatory models (DSS, C. rodentium, T gondii) in both male and female mice, in order to evaluate how the sensors function and express violacein when inflammation is detected in the small intestine and/or colon. For these studies, mice may be given oral gavage of 10⁹ biosensors each morning during the DSS/infection period, and fecal pellets may be collected throughout the days. Colonic, cecal, and small intestinal contents may also be collected to evaluate upstream pigment shifts and possible localized signal detection. Chromatographic shift of fecal color may be observed in order to ascertain if violacein can be detected visually in the stool. Violacein may be extracted in an established ethanol-based procedure to measure via spectrophotometry.

In at least some cases one can utilize in vitro, ex vivo, and/or in vivo methods, in order to evaluate efficacy and specificity of the synthetic E. coli biosensors. Observable production in the presence of purified calprotectin and no baseline production in the absence of calprotectin may occur. One can further characterize the biosensors using MBRA and in vivo validation. Colorimetric shifts to both calprotectin-positive MBRA broth media and inflamed mouse fecal pellets, particularly visible without further experimental evaluation, may be utilized as a viable diagnostic for gastrointestinal inflammation. In particular embodiments there is a sustained signal from small intestinal inflammation detection through to excretion.

In specific cases, one can utilize positive feedback loops, such as the LuxI/LuxR quorum sensing unit, to sustain violacein output in response to calprotectin-sensing [26]. LuxR positively autoregulates itself, so luxR induction via calprotectin sensing can turn the Lux system on and then direct sustained violacein production by placing the lux promoter upstream of both the reporter gene and an additional luxR.

### Example 2

### Microbial Biosensors for Intestinal Inflammation

### Examples of Methods

Minimum inhibitory concentration (MIC) assay—Escherichia coli Nissle 1917 and Lactobacillus reuteri PTA 6475 were used as vectors for biosensor construction. For both strains, bacteria was grown overnight in media (MRS broth for L. reuteri; LB broth for E. coli). From the overnight culture, 10\hat{ }4 bacterial cells in 38 uL media (LDM4 for L. reuteri; LB broth for E. coli) were seeded into a 96 well-plate. 62 uL of calprotectin in buffer (20 mM Tris (pH 7.5), 100 mM NaCl, 10mM beta-mercaptoethanol, 3 mM CaCl2) was added in 2-fold dilutions. Recombinant human calprotectin was supplied by Dr. Walter Chazin of Vanderbilt University. Bacteria was allowed to grow overnight, and OD600 values were taken the following morning.

RNA Isolation—Sub-inhibitory concentrations of calprotectin were used for the calprotectin induction. These values were the highest concentration of calprotectin that did not inhibit overnight growth (15.625 ug/mL for L. reuteri, 125 ug/mL for E. coli Nissle). Cells were grown overnight, and then back-diluted 1:100 into 5 mL of media (LDM4 for L. reuteri; LB Broth for E. coli). Cells were grown up to log phase, and then the sub-inhibitory amount of calprotectin was added to the tube in calprotectin buffer. Growth continued for 30 total minutes in the presence of the calprotectin, and then transcription was frozen by addition of ice cold methanol for L. reuteri and ice-cold ethanol for E. coli Nissle. Cells were collected via centrifugation, and then RNA was isolated using the QIAgen RNEasy kit. Slight modifications were included. For L. reuteri, mechanical cell-wall disruption was used via bead-beating. For E. coli, lysozyme/proteinase K was used to disrupt the gram-negative cell wall. RNA was quantified and tested for purity using a Nanodrop.

RNA-seq—RNA-seq was performed by Applied Biological Materials (ABM) (Richmond, British Columbia, Canada) on an Illumina NextSeq sequencer, at an average of 5 million reads per sample. rRNA depletion and quality check was also performed by ABM. Sequences were received from ABM in fastq format.

Transcriptome analysis—Raw sequence filtering and alignment was performed by the Center for Metagenomics and Microbiome Research at Baylor College of Medicine. Sequences were aligned against L. reuteri 6475/MM4-1A and E. coli Nissle 1917 reference genomes. Raw counts were obtained and gene expression analysis was performed using DESeq2 on R-Studio. Genes that had at least a 2-fold increase in expression in the presence of calprotectin were passed for construct engineering.

Construct Engineering—Using the aforementioned reference genomes, upstream intergenic promoter regions were identified for a collection of gene clusters upregulated at least 2-fold by calprotectin for both E. coli Nissle and L. reuteri. DNA was isolated from both strains and promoter regions were amplified via PCR.

Plasmids—The pColE1 plasmid was used for all E. coli Nissle constructs and a PSIP411 derivative that lacks the sakacin inducible system was used for L. reuteri constructs.

Initial test constructs consisted of the intergenic promoter regions for the following genes: E. coli Nissle—sulA, entCE (enterobactin synthase operon), ykgMO (paralog for L36/L31 ribosomal accessory protein), ABC Transporter (WP 000977398.1); L. reuteri—ABC Transporter operon (EGC15836.1), Acyl carrier protein dehydratase (EGC15031.1), argininosuccinate lyase (EGC15288.1). Each promoter region was cloned directly upstream of a green fluorescent protein cassette. For L. reuteri, the eGFP cassette was used while the sGFP cassette was used for E. coli. All constructs were assembled via the Gibson Reaction (NEBiosciences). All Gibson oligos were made using IDT, and all amplicons were synthesized with Phusion polymerase (NEBiosciences).

Competent cells were made fresh for each transformation. Electroporation was the sole method of transformation. All E. coli Nissle were cloned directly into E. coli Nissle 1917 following Gibson assembly. All L. reuteri constructs were first cloned into E. coli EC1000, and after sequence confirmation, were then ported into L. reuteri PTA6475.

Cell culture for flow analysis—Upon sequence confirmation of final constructs, cells were grown to early logarithmic phase (-0.1 OD600) and frozen in 15% glycerol with media. All flow runs used these cells frozen at early log phase. All E. coli Nissle gfp constructs were grown in M9 media with glucose and all L. reuteri constructs were grown in LDM4 minimal media so as to reduce possible cross-effects on gfp expression. All flow runs were performed in 96 well plates.

Calprotectin Induction—The following mix was added to each well—124 uL of recombinant human calprotectin at 20-80 ug/mL in calprotectin buffer, 56 uL media (M9 for E. coli and LDM4 for L. reuteri), and 20 uL of cells. For L. reuteri, 2 uL of additional MRS was added to aid in L. reuteri growth.

E. coli Nissle was grown for ˜4 hours aerobically, until OD600 reached 0.10-0.15, for a total of ˜5 cell divisions. L. reuteri was grown for ˜5-6 hours anaerobically, until OD600 reached 0.15, for a total of ˜5 cell divisions. At this point, cells were kept on ice until analysis on a BD FACScan flow cytometer.

TPEN Induction—TPEN, a synthetic zinc chelator, induction assays were performed as calprotectin induction, with TPEN replacing calprotectin. Growth condition were also identical. Media ratios differed. In the TPEN studies, 180 uL of media was used with 20 uL cells and 2 uL of TPEN in absolute ethanol.

Metal complementation assays—Mixes were similar to the calprotectin induction assays with the addition of zinc (II) sulfate, manganese (II) sulfate, or iron (II) chloride. Concentrations of metals were added in excess of 1×, 10×, and 100× the binding capacity of 40 ug/mL (1.4 uM) calprotectin. In total, 0, 4 uM, 40 uM, and 400 uM of zinc and iron were added and 0, 2 uM, 20 uM, and 200 uM of manganese was added. Slightly higher than equimolar amounts were added to correct for possible pipetting error. Cells were grown for the same amount of time as in the calprotectin induction assays.

Calprotectin ELISA—IBD and healthy fecal samples were procured from Dr. Richard Kellermayer. The Immundiagnostik IDK Calprotectin ELISA was used to evaluate calprotectin concentrations in the fecal samples. Manufacturer's instructions were followed. Samples were diluted as necessary to fit the standard curve.

IBD Sample Induction—IBD samples were diluted 1:1 (100 milligrams of fecal matter in 100 uL PBS), and then mixed and vortexed vigorously. Slurries were centrifuged for 5 minutes at 14K RPM, and supernatants were separated and used for the flow runs. The following mix was added to each well—160 uL media (M9 for E. coli and LDM4 for L. reuteri), 20 uL of cells, and 20 uL of fecal slurry.

Growth conditions were identical to the calprotectin induction methods. Upon reaching early logarithmic phase OD, cells were kept on ice until analysis on flow.

Flow cytometry—˜10-40 uL of cells were added to 1 mL of PBS sheath fluid and run through the flow cytometer for a total of 10000 events. Cells were thresholded by forward/side scatter, and .fsc files were analyzed with the FlowCal software, developed by the Jeff Tabor Lab. In short, FlowCal identifies the densest region of cells on the associated scatterplot, and analyzes the fluorescent output of 30% of the cells in this region so as to evaluate a homogenous dataset and remove possible outliers. This results in a geometric mean of total fluorescent output. In this case, GFP output is reported as molecules of equivalent fluorophores (MEF), and was evaluated on the FL1 channel. More information on FlowCal can be found at: http//taborlab.github.io/FlowCal/

### Examples of Results

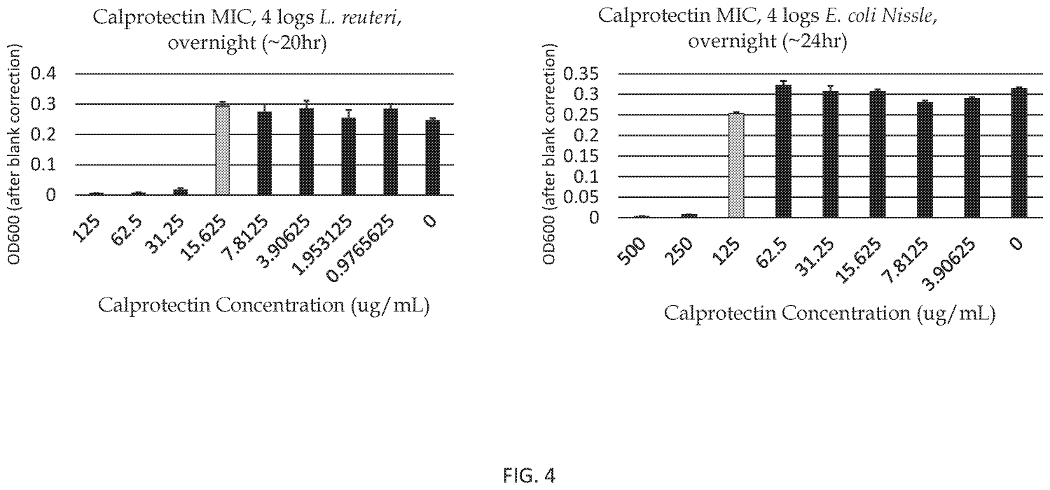

Both prospective vector strains (Lactobacillus reuteri and Escherichia coli Nissle) were grown overnight in the presence of 2-fold dilutions of calprotectin in order to deduce minimum inhibitory concentrations of calprotectin on the microbes. L. reuteri growth was inhibited at 31.25 ug/mL of calprotectin and above, while E. coli Nissle growth was inhibited at 250 ug/mL of calprotectin and above (FIG. 4).

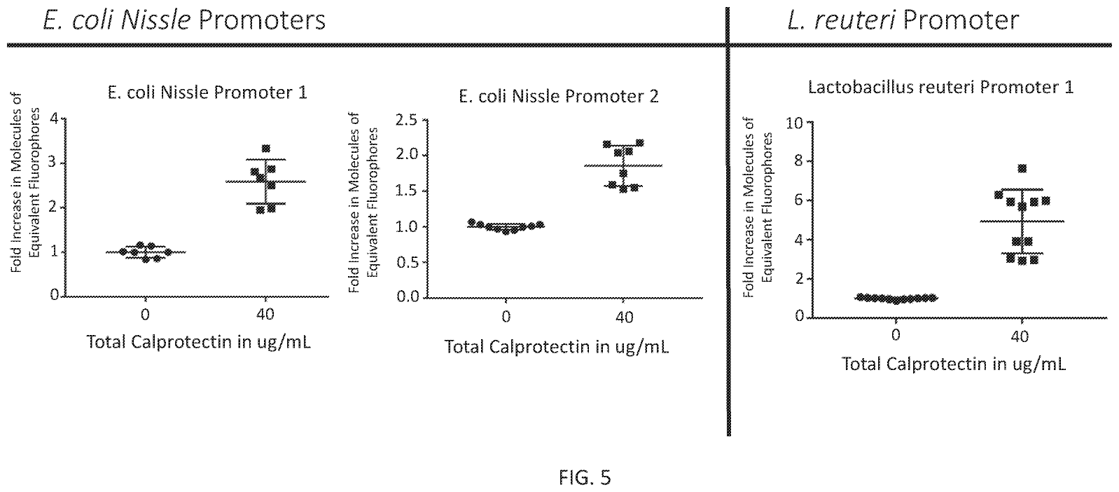

Two E. coli Nissle and one L. reuteri biosensors exhibited increased GFP expression when in the presence of 40 ug/mL of calprotectin. Calprotectin induced L31/L36 ribosomal accessory paralog promoter-driven GFP expression 2.6-fold higher than L31/L36 cells grown without calprotectin. Likewise, the enterobactin synthase promoter biosensor exhibited a 1.8-fold increase in GFP expression when co-cultured with calprotectin. The Lactobacillus reuteri ABC transporter promoter sensor increased GFP expression 4.93-fold over uninduced sensor (FIG. 5).

The biosensors comprising calprotectin-sensitive promoters are utilized against a cohort of IBD and healthy fecal slurries. Biosensors that are co-cultured in fecal slurries containing >100 (or 125, 150, 175, 200) ug/mL calprotectin express significantly increased GFP compared to sensors co-cultured in slurries containing less than 100 μg/ml of calprotectin, in at least some cases.

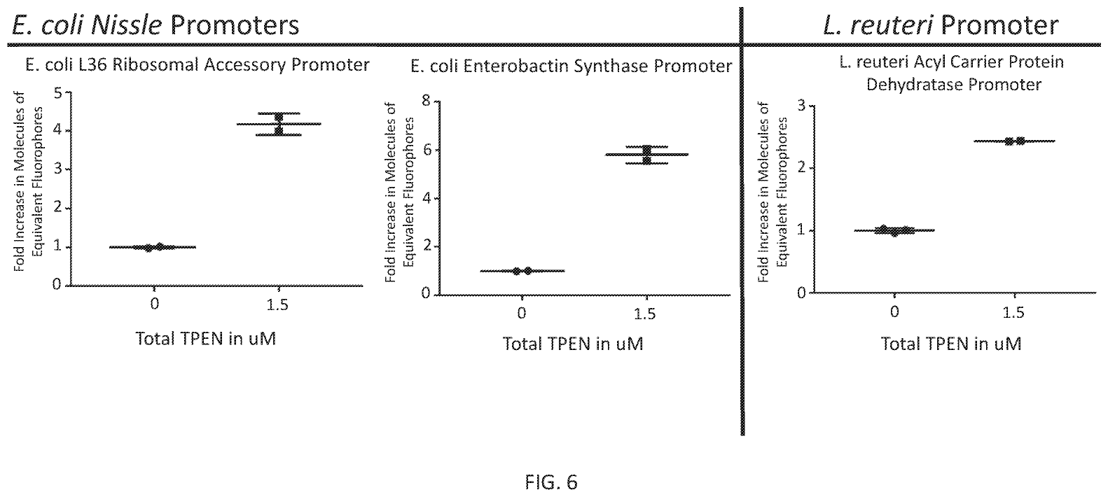

Biosensors grown in the presence of the synthetic zinc chelator, TPEN, also demonstrated an increase in GFP expression. GFP expression in L36 ribosomal accessory promoter biosensor co-cultured with TPEN was increased 4.5-fold over sensors grown in control media, and was also increased 5.6-fold in the enterobactin synthase promoter biosensor after TPEN induction. The L. reuteri acyl carrier protein dehydratase promoter biosensor exhibited a 2.5-fold increase in GFP expression when co-cultured with TPEN, compared with the uninduced sensor (FIG. 6).

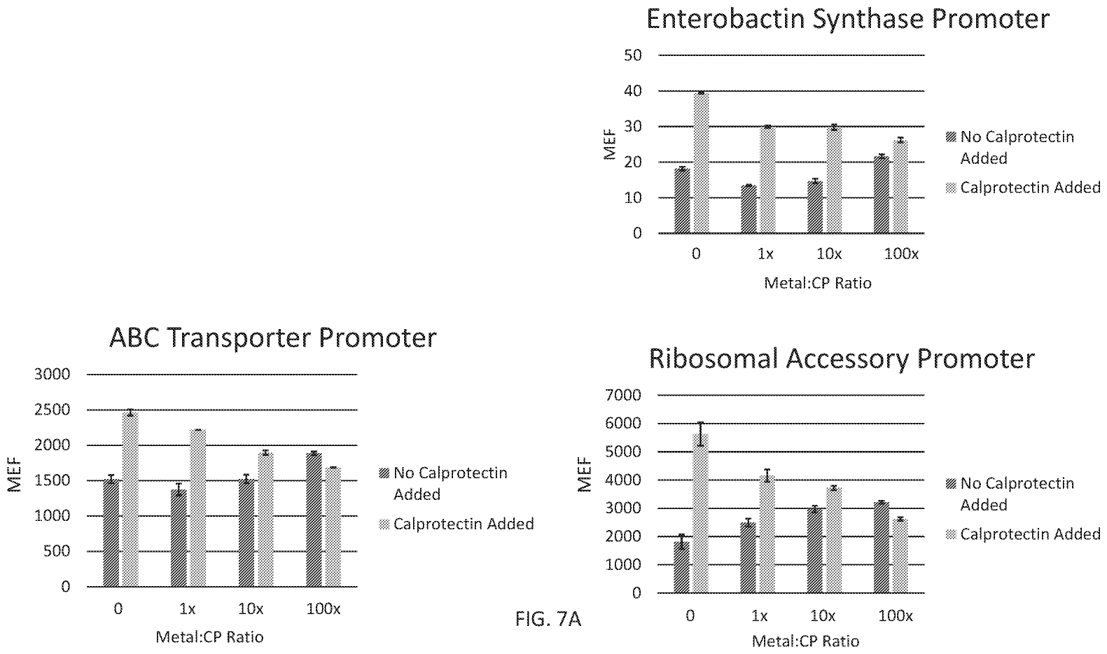

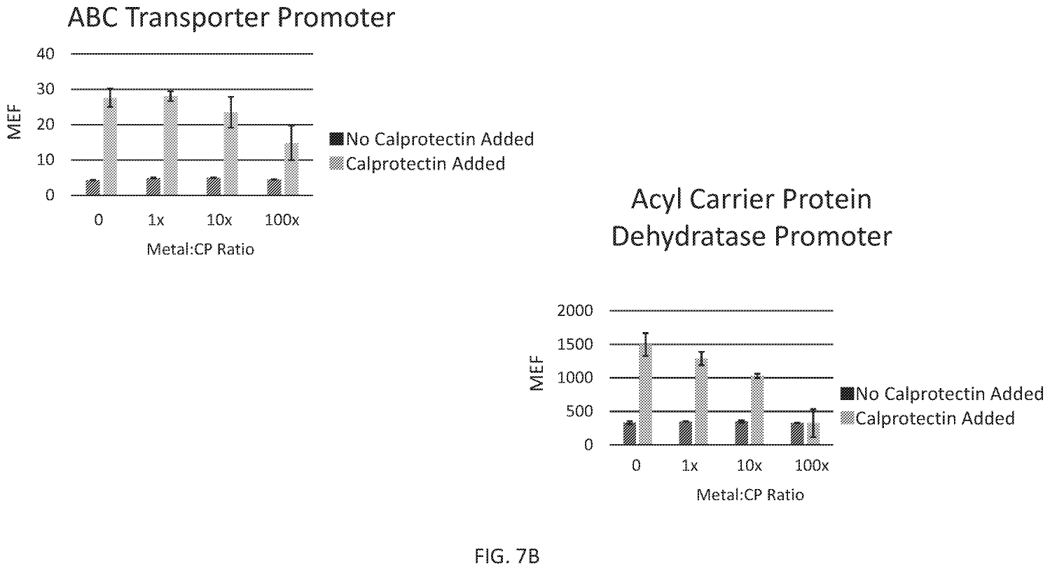

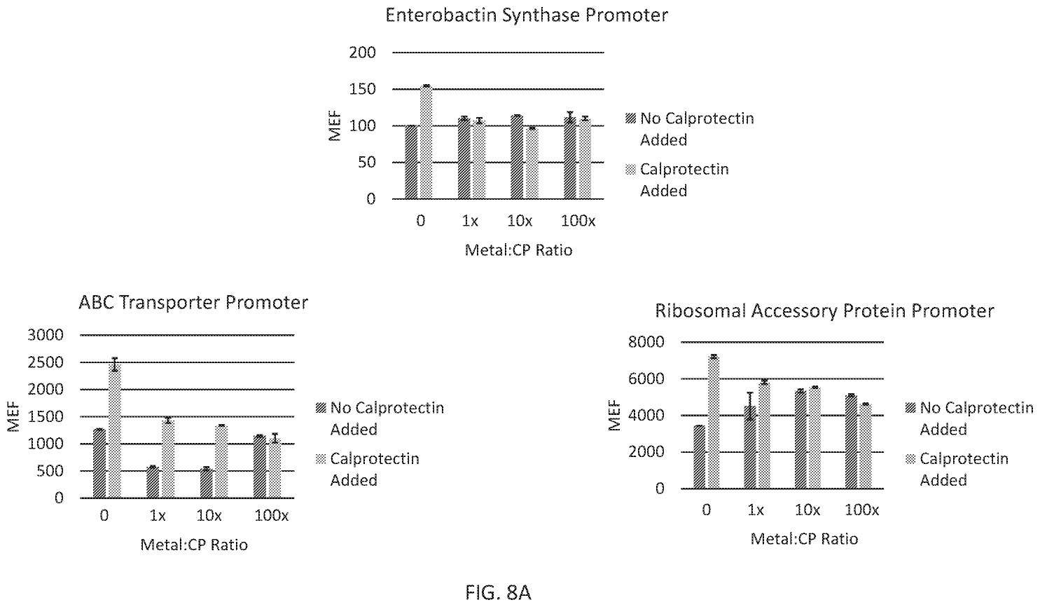

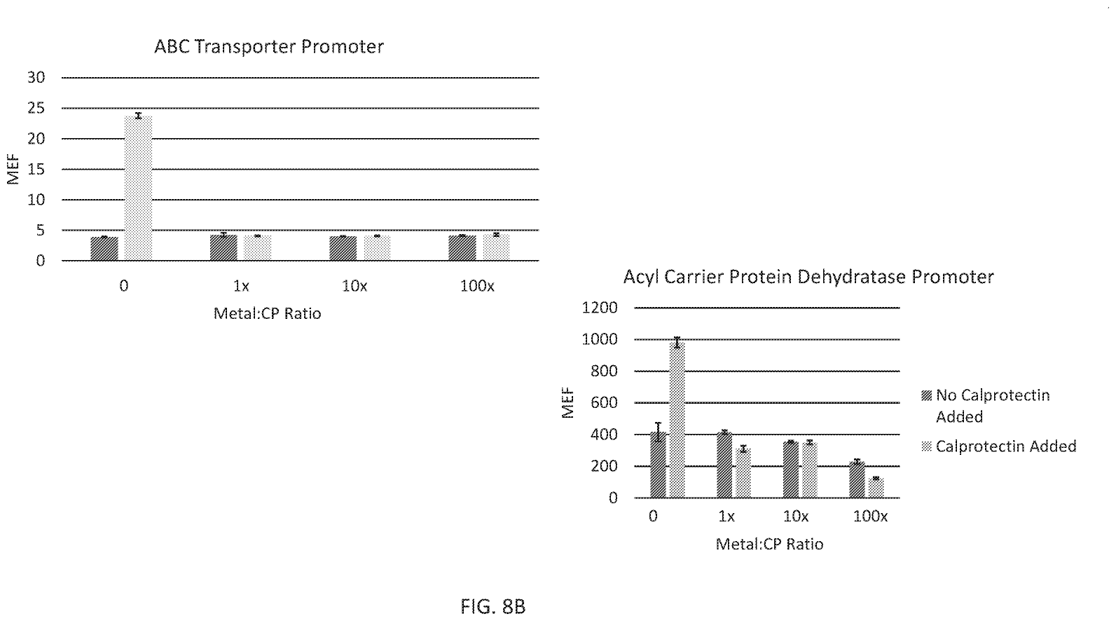

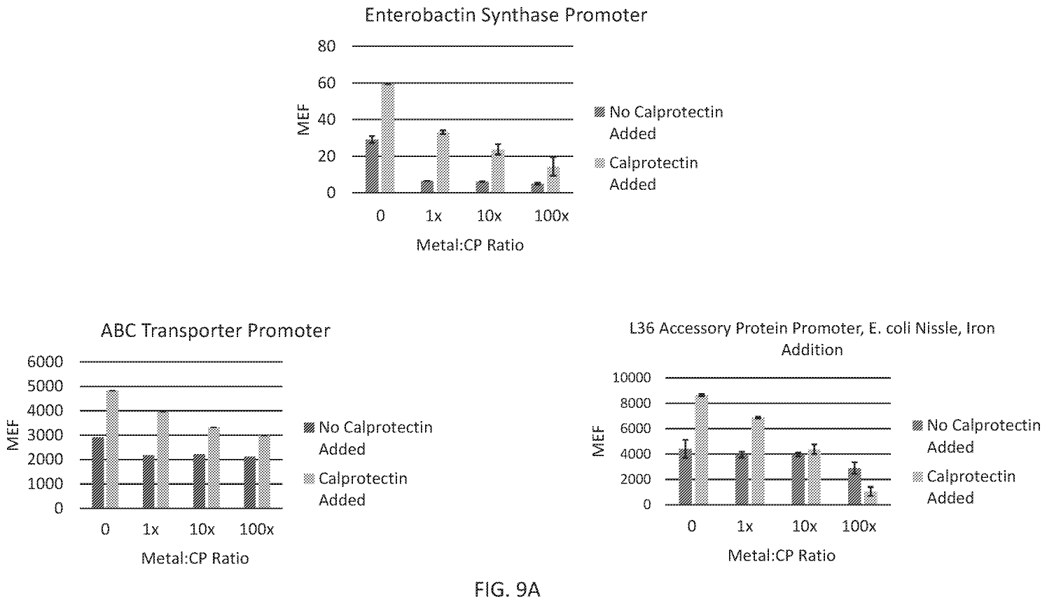

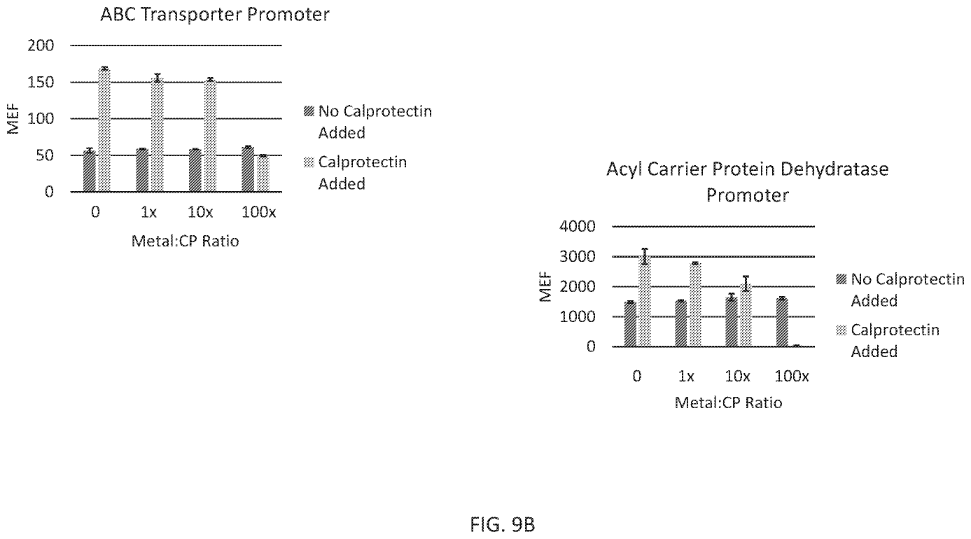

Free metal complementation results varied based on metal and promoter construct. Even at 100× binding capacity, manganese did not fully ablate sensor activity in the L. reuteri ABC transporter biosensor (FIGS. 7A and 7B). Lower levels of zinc addition was able to shut off the sensors. 4 uM zinc was sufficient to totally abolish enterobactin synthase promoter activity, as well as the L. reuteri ABC transporter promoter and the acyl carrier protein dehydratase promoter (FIGS. 8A and 8B). As with manganese, iron was varied in its effects upon the promoter constructs. 100× iron was required to turn off L. reuteri ABC transporter promoter activity, though this could also be a cell survival issue, considering the toxicity of high levels of iron (FIGS. 9A and 9B).

### REFERENCES

1. Brown S J, Mayer L (2007) The immune response in inflammatory bowel disease. Am J Gastroenterol 102: 2058-2069.

2. Romberg-Camps M J, Bol Y, Dagnelie P C, Hesselink-van de Kruijs M A, Kester A D, et al. (2010) Fatigue and health-related quality of life in inflammatory bowel disease: results from a population-based study in the Netherlands: the IBD-South Limburg cohort. Inflamm Bowel Dis 16: 2137-2147.

3. Roseth A G, Schmidt P N, Fagerhol M K (1999) Correlation between faecal excretion of indium-111-labelled granulocytes and calprotectin, a granulocyte marker protein, in patients with inflammatory bowel disease. Scand J Gastroenterol 34: 50-54.

4. Nakashige T G, Zhang B, Krebs C, Nolan E M (2015) Human calprotectin is an iron-sequestering host-defense protein. Nat Chem Biol 11: 765-771.

5. Damo S M, Kehl-Fie T E, Sugitani N, Holt M E, Rathi S, et al. (2013) Molecular basis for manganese sequestration by calprotectin and roles in the innate immune response to invading bacterial pathogens. Proc Natl Acad Sci USA 110: 3841-3846.

6. Clohessy P A, Golden B E (1995) Calprotectin-mediated zinc chelation as a biostatic mechanism in host defence. Scand J Immunol 42: 551-556.

7. Walsham N E, Sherwood R A (2016) Fecal calprotectin in inflammatory bowel disease. Clin Exp Gastroenterol 9: 21-29.

8. Lehmann F S, Burri E, Beglinger C (2015) The role and utility of faecal markers in inflammatory bowel disease. Therap Adv Gastroenterol 8: 23-36.

9. Maréchal C, Aimone-Gastin I, Baumann C, Dirrenberger B, Guéant J-L, et al. Compliance with the faecal calprotectin test in patients with inflammatory bowel disease. United European Gastroenterology Journal 0: 2050640616686517.

10. Daeffler K N, Galley J D, Sheth R U, Ortiz-Velez L C, Shroyer N F, et al. (2017) Engineering bacterial thiosulfate and tetrathionate sensors for detecting gut inflammation. Molecular

[symbol]

11. Danino T, Prindle A, Kwong G A, Skalak M, Li H, et al. (2015) Programmable probiotics for detection of cancer in urine. Sci Transl Med 7: 289ra284.

12. Courbet A, Endy D, Renard E, Molina F, Bonnet J (2015) Detection of pathological biomarkers in human clinical samples via amplifying genetic switches and logic gates. Sci Transl Med 7: 289ra283.

13. Choi H J, Ahn J H, Park S H, Do K H, Kim J, et al. (2012) Enhanced wound healing by recombinant Escherichia coli Nissle 1917 via human epidermal growth factor receptor in human intestinal epithelial cells: therapeutic implication using recombinant probiotics. Infect Immun 80: 1079-1087.

14. Seo E J, Weibel S, Wehkamp J, Oelschlaeger T A (2012) Construction of recombinant E. coli Nissle 1917 (EcN) strains for the expression and secretion of defensins. Int J Med Microbiol 302: 276-287.

15. Pawlik M C, Hubert K, Joseph B, Claus H, Schoen C, et al. (2012) The zinc-responsive regulon of Neisseria meningitidis comprises 17 genes under control of a Zur element. J Bacteriol 194: 6594-6603.

16. Shin J H, Helmann J D (2016) Molecular logic of the Zur-regulated zinc deprivation response in Bacillus subtilis. Nat Commun 7: 12612.

17. Murphy K F, Balazsi G, Collins J J (2007) Combinatorial promoter design for engineering noisy gene expression. Proc Natl Acad Sci USA 104: 12726-12731.

18. Badran A H, Guzov V M, Huai Q, Kemp M M, Vishwanath P, et al. (2016) Continuous evolution of Bacillus thuringiensis toxins overcomes insect resistance. Nature 533: 58-63.

19. Wang B, Barahona M, Buck M (2015) Amplification of small molecule-inducible gene expression via tuning of intracellular receptor densities. Nucleic Acids Res 43: 1955-1964.

20. Auchtung J M, Robinson C D, Britton R A (2015) Cultivation of stable, reproducible microbial communities from different fecal donors using minibioreactor arrays (MBRAs). Microbiome 3: 42.

21. Barman T K, Rao M, Bhati A, Kishore K, Shukla G, et al. (2011) Non invasive real-time monitoring of bacterial infection & therapeutic effect of anti-microbials in five mouse models. Indian J Med Res 134: 688-695.

22. Nighot P, Al-Sadi R, Rawat M, Guo S, Watterson D M, et al. (2015) Matrix metalloproteinase 9-induced increase in intestinal epithelial tight junction permeability contributes to the severity of experimental DSS colitis. Am J Physiol Gastrointest Liver Physiol 309: G988-997.

23. Mackos A R, Galley J D, Eubank T D, Easterling R S, Parry N M, et al. (2016) Social stress-enhanced severity of Citrobacter rodentium-induced colitis is CCL2-dependent and attenuated by probiotic Lactobacillus reuteri. Mucosal Immunol 9: 515-526. PMCID-PMC4794400

24. Dias R R, Carvalho E C, Leite C C, Tedesco R C, Calabrese Kda S, et al. (2014) Toxoplasma gondii oral infection induces intestinal inflammation and retinochoroiditis in mice genetically selected for immune oral tolerance resistance. PLoS One 9: e113374.

25. Coombes J L, Charsar B A, Han S J, Halkias J, Chan S W, et al. (2013) Motile invaded neutrophils in the small intestine of Toxoplasma gondii-infected mice reveal a potential mechanism for parasite spread. Proc Natl Acad Sci USA 110: E1913-1922.

26. Sayut D J, Niu Y, Sun L (2006) Construction and engineering of positive feedback loops. ACS Chem Biol 1: 692-696.

27. Blosser R S, Gray K M (2000) Extraction of violacein from Chromobacterium violaceum provides a new quantitative bioassay for N-acyl homoserine lactone autoinducers. J Microbiol Methods 40: 47-55.

28. Jiang P X, Wang H S, Zhang C, Lou K, Xing X H (2010) Reconstruction of the violacein biosynthetic pathway from Duganella sp. B2 in different heterologous hosts. Appl Microbiol Biotechnol 86: 1077-1088.

29. Zhao X, Shi F, Zhan W (2015) Overexpression of ZWF1 and POSS improves carotenoid biosynthesis in recombinant Saccharomyces cerevisiae. Lett Appl Microbiol 61: 354-360.

30. Bhushan B, Samanta S K, Jain R K (2000) Indigo production by naphthalene-degrading bacteria. Lett Appl Microbiol 31: 5-9.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps. Moreover, the scope of the present application is not intended to be limited to the particular embodiments of the process, machine, manufacture, composition of matter, means, methods and steps described in the specification.

1. A method of determining a need for therapy for intestinal inflammation or cancer in an individual, comprising the steps of:
providing to the individual a population of non-pathogenic bacteria comprising an engineered polynucleotide, said polynucleotide comprising one or more direct calprotectin-sensor sequences or indirect calprotectin-sensor sequences operably linked to expression of a detectable readout product; and examining the feces of said individual for the detectable readout product.

2. The method of claim 1, wherein the calprotectin-sensor sequence is a bacterial promoter.

3. The method of claim 1, wherein the sensor sequence comprises part or all of one or more promoters from Lactobacillus reuteri 6475 and Escherichia coli Nissle 1917.

4. The method of claim 1, wherein the calprotectin-sensor sequence comprises part or all of the L36/L31 ribosomal accessory protein promoter, part or all of the promoter for the enterobactin synthase, or both.

5. The method of claim 1, wherein the indirect calprotectin-sensor sequences are directly or indirectly sensitive to a metal to which calprotectin binds.

6. The method of claim 5, wherein the metal is free zinc, iron, manganese, or a combination thereof.

7. The method of claim 1, wherein the readout product is a detectable colorimetric or fluorescent marker.

8. The method of claim 1, wherein the readout product is one or more of the following: violacein, one or more carotenoids, one or more phycobilins, one or more anthocyanins, and indigo.

9. The method of claim 1, wherein the readout product is green fluorescence protein, yellow fluorescent protein, blue fluorescent protein, mCherry, or cyan fluorescent protein.

10. The method of claim 1, wherein the providing step occurs orally.

11. The method of claim 1, wherein the providing step is performed by the individual.

12. The method of claim 1, wherein the providing step occurs on a regular basis.

13. The method of claim 1, wherein the providing step occurs during the presence or absence of one or more symptoms of intestinal inflammation.

14. The method of claim 1, wherein when the level of calprotectin in the gastrointestinal tract of the individual is ≥100 ug/mL, the readout product is detectable.

15. The method of claim 1, wherein the examining step of the feces for the detectable readout product is performed by the individual.

16. The method of claim 1, wherein the calprotectin-sensor sequence comprises one or more zinc-uptake-regulator sites.

17. The method of claim 1, wherein when the readout product is detected, the individual obtains treatment for the intestinal inflammation.

18. The method of claim 1, wherein the intestinal inflammation is from inflammatory bowel disease (IBD).

19. The method of claim 18, wherein the IBD is Crohn's Disease (CD) or ulcerative colitis (UC).

20. The method of claim 1, wherein when the readout product is detected, the individual receives treatment of the inflammation or cancer prior to onset of one or more symptoms.

21. A non-pathogenic bacteria comprising an engineered polynucleotide, said polynucleotide comprising one or more direct calprotectin-sensor sequences or indirect calprotectin-sensor sequences operably linked to a sequence that encodes a detectable readout product.

22. The bacteria of claim 21, wherein the calprotectin-sensor sequence is a bacterial promoter.

23. The bacteria of claim 21, wherein the sensor sequence comprises part or all of one or more promoters from Lactobacillus reuteri 6475 and Escherichia coli Nissle 1917.

24. The bacteria of claim 21, wherein the calprotectin-sensor sequence comprises part or all of the L36/L31 ribosomal accessory protein promoter, part or all of the promoter for the enterobactin synthase, or both.

25. The bacteria of claim 21, wherein the indirect calprotectin-sensor sequences are directly or indirectly sensitive to a metal to which calprotectin binds.

26. The bacteria of claim 25, wherein the metal is free zinc, iron, manganese, or a combination thereof.

27. The bacteria of claim 21, wherein the readout product is a detectable colorimetric or fluorescent marker.

28. The bacteria of claim 21, wherein the readout product is one or more of the following: violacein, one or more carotenoids, one or more phycobilins, one or more anthocyanins, and indigo.

29. The bacteria of claim 21, wherein the readout product is green fluorescence protein, yellow fluorescent protein, blue fluorescent protein, one or more phycobilins, one or more anthocyanins, or cyan fluorescent protein.


SEQUENCE LISTING (5 sequences)

SEQ ID NO: 1 - DNA, 364 bp, Escherichia coli
taacggcaataaactgttcacttcagtgatatttaaaatatgcatcctctcccttttttgtaagtaattattatatccgtgggagaggaatacacattgtcaggtaatcaatcatgctgcaataaatcatcggccagtaaagtggagatagcctccattctcgaaaaatccatactctcagcgaaaccatcatcaatcactcatccaggcgtttatgggagcgtcgccaatggctgctaacaatgccagacttccccgttgcggaaattccacatcccacaaatagtcacagtgattgggtgttgaaatgatccggatgagcatgtatctttacggttatgttataacataacaggtaaaaatg

SEQ ID NO: 2 - DNA, 188 bp, Escherichia coli
aagtcagcttcctgttattaatgaagttaatgcttctcattttcattgtaaccacaaccagatgcaaccccgagttgcagattgcgttacctcaagagttgacatagtgcgcgtttgcttttaggttagcgaccgaaaatataaatgataatcattattaaagcctttatcattttgtggaggatgat

SEQ ID NO: 3 - DNA, 336 bp, Escherichia coli
aacagacccgaagaaaatgaaatataagaaaagatcaacgagtgaagaaaaagttcaaaaaatggctgccggggaggaaggaaagtaccggatggaaagagtcccccctaaagcagactgacagacataacaaatccccggggggatttgtgtataagagacagtacttatctggaggtaattgcaatatctctgtgaacttacacagggtgggcttaccgcatacactgacacttagcggatcgacagaacattattaacagagcatcactgaacgctacataatcagagttgcataaataaaatgttattaacatcacaatcacaacatttcga

SEQ ID NO: 4 - DNA, 182 bp, Lactobacillus reuteri
ttaataacgagcaaatattattttaccaattctcaattaaatagtaaaggaatattttaatttaaggtgattaagaattaaatgacataaaaataatttgacattcaaaatatttttgaatataatttgatcatcgaatattttgatactcgaaatatttttttgaaggcaggtgaatcttt

SEQ ID NO: 5 - DNA, 139 bp, Lactobacillus reuteri
tcatatttaaggacaagaggatggaaagaagtaactttctctcttgtcctttttatttttatgttgcaatcccgcataaagttcgatattatattcattgttctaaatagtaacgattacgattaaaaagagggaaaga